(19) United States
(12) Patent Application Publication
(10) Pub. No.: US 2026/0157864 A1
(43) Pub. Date: Jun. 11, 2026

(54) APPARATUS AND METHOD FOR ENGAGING PROJECTILES

(71) Applicant: AcceliPHI, LLC, Rome, NY (US)

(72) Inventors: Evan M. Drozd, Rome, NY (US); Andrew Louis Drozd, Rome, NY (US); Barbara Ann Welch-Drozd, Rome, NY (US)

(21) Appl. No.: 18/969,486

(22) Filed: Dec. 5, 2024

(51) Int. Cl. A61F 2/58; A61F 2/50; A61F 2/68; A61F 2/76

(52) U.S. Cl. CPC A61F 2/586; A61F 2/68; A61F 2/76; A61F 2002/5021; A61F 2002/6836

(57) ABSTRACT

An apparatus and method for engaging projectiles. The apparatus includes a digit configured to operably engage a projectile. The digit includes a plurality of phalanx interdigitated with a respective set of spacers and a deformable material externally coupled to at least one of the plurality of phalanx. The digit is coupled to an adjustable fastener securing the digit to a user's residual limb. Further, an articulating mechanism is coupled to the plurality of phalanx and can adjust the position of the digit.

### TECHNICAL FIELD

Embodiments of the disclosure relate generally to prosthetic devices and, more specifically, to an apparatus and method structured for restoring ability to participate in certain physical activities.

### BACKGROUND

Prosthetic devices, particularly limbs, may be structured to help survivors of an injury restore their ability to participate in certain activities, including sports. In the case of sports requiring interaction with a projectile (e.g., a bowling ball, a soccer ball, a basketball, rachet and disc, etc.) physical interaction between the projectile and each digit of a user's hand significantly affects performance. Each individual digit serves a different role in supporting the projectile, such as contributing to control of force applied to the projectile (e.g. axis tilt, revolution rate, and trajectory). Unfortunately, conventional prosthetic devices have proven unsatisfactory in replicating the exact motion of specific digits. Without an appropriate prosthetic device, injuries to a user's digit(s) can affect technique, performance, and overall experience. Conventional prosthetics continue to hinder the user's ability to control, release, and impart rotational force on the projectile, or conversely, provide similarly undesirable competitive advantages against non-injured participants.

### BRIEF SUMMARY

All aspects, examples and features mentioned below can be combined in any technically possible way.

Aspects of the disclosure include an apparatus comprising a digit configured to operably engage a projectile, the digit comprising: a plurality of phalanx interdigitated with a respective set of spacers and configured to replicate the articulation of a counterpart digit, and a deformable material externally coupled to one of the plurality of phalanx configured to replicate a grip of the counterpart digit on the projectile; an adjustable fastener coupled to the digit and configured to securing the digit to a limb; and an articulating mechanism operatively coupled to the plurality of phalanx and configured to adjust a positional configuration of the digit.

Another aspect of the disclosure includes any of the preceding aspects, wherein a length of the digit is proportional to a partially amputated finger of a patient.

Another aspect of the disclosure includes any of the preceding aspects, wherein the adjustable fastener includes: a mechanical fastener configured to secure the digit to the limb, and a sleeve configured to mate with the partially amputated finger of the patient and connect to the mechanical fastener through a set of anchoring wires.

Another aspect of the disclosure includes any of the preceding aspects, wherein a length of the digit is proportional to a fully amputated finger of a patient.

Another aspect of the disclosure includes any of the preceding aspects, wherein the adjustable fastener is a dorsal hand plate including: a first plate coupled to the digit and operatively engaging a second plate, a well having the second plate therein; and a mechanical fastener coupling the well to the limb.

Another aspect of the disclosure includes any of the preceding aspects, wherein a set of interlocked teeth couple the first plate to the second plate.

Another aspect of the disclosure includes any of the preceding aspects, wherein the deformable material is coupled to an exterior of at least two phalanx of the plurality of phalanx.

Further aspects of the disclosure include an apparatus comprising a digit configured to operably engage a projectile, the digit comprising: a plurality of phalanx interdigitated with a respective set of spacers and configured to replicate the articulation of a counterpart digit, and a deformable material externally coupled to one of the plurality of phalanx configured to replicate a grip of the counterpart digit on the projectile; an adjustable fastener coupled to the digit and configured to securing the digit to a limb; an articulating mechanism operatively coupled to the plurality of phalanx and configured to adjust a positional configuration of the digit; and a controller connected to the articulating mechanism and configured to operate the articulating mechanism based on a force applied to the projectile.

Another aspect of the disclosure includes any of the preceding aspects, wherein a length of the digit is proportional to a partially amputated finger of a patient.

Another aspect of the disclosure includes any of the preceding aspects, wherein the adjustable fastener includes: a mechanical fastener configured to secure the digit to the limb, and a sleeve configured to mate with the partially amputated finger of the patient and connect to the mechanical fastener through a set of anchoring wires.

Another aspect of the disclosure includes any of the preceding aspects, wherein a length of the digit is proportional to a fully amputated finger of a patient.

Another aspect of the disclosure includes any of the preceding aspects, wherein the adjustable fastener is a dorsal hand plate including: a first plate coupled to the digit and operatively engaging a second plate, a well having the second plate therein; and a mechanical fastener coupling the well to the limb.

Another aspect of the disclosure includes any of the preceding aspects, wherein a set of interlocked teeth couple the first plate to the second plate.

Another aspect of the disclosure includes any of the preceding aspects, wherein the deformable material is coupled to an exterior of at least two phalanx of the plurality of phalanx.

Another aspect of the disclosure includes any of the preceding aspects, wherein the articulating mechanism comprises a gear set disposed within the set of spacers.

Further aspects of the disclosure include a method of manufacturing an apparatus, the method comprising: forming a digit configured to operably engage a projectile, the digit including: a plurality of phalanx interdigitated with a respective set of spacers and configured to replicate the articulation of a counterpart digit, and a deformable material externally coupled to one of the plurality of phalanx configured to replicate a grip of the counterpart digit on the projectile; forming an adjustable fastener coupled to the digit and configured to securing the digit to a limb; and forming an articulating mechanism operatively coupled to the plurality of phalanx and configured to adjust a positional configuration of the digit.

Another aspect of the disclosure includes any of the preceding aspects, wherein a length of the digit is formed proportionally to a partially amputated finger of a patient.

Another aspect of the disclosure includes any of the preceding aspects, wherein the adjustable fastener includes: a mechanical fastener configured to secure the digit to the limb, and a sleeve configured to mate with the partially amputated finger of the patient and connect to the mechanical fastener through a set of anchoring wires.

Another aspect of the disclosure includes any of the preceding aspects, wherein a length of the digit is formed proportionally to a fully amputated finger of a patient.

Another aspect of the disclosure includes any of the preceding aspects, wherein the adjustable fastener is a dorsal hand plate including: a first plate coupled to the digit and operatively engaging a second plate, a well having the second plate therein; and a mechanical fastener coupling the well to the limb.

Two or more aspects described in this disclosure, including those described in this summary section, may be combined to form implementations not specifically described herein. That is, all embodiments described herein can be combined with each other.

The details of one or more implementations are set forth in the accompanying drawings and the description below. Other features, objects and advantages will be apparent from the description and drawings, and from the claims.

### BRIEF DESCRIPTION OF THE DRAWINGS

The drawings illustrate embodiments presently contemplated for carrying out the invention. In the drawings:

FIG. 1. shows an perspective view of an embodiment of an apparatus according to embodiments of the disclosure;

FIG. 2 shows a dorsal view of an apparatus configured for use with a full amputation according to embodiments of the disclosure;

FIG. 3A shows a side view of a dorsal hand plate in an apparatus according to embodiments of the disclosure;

FIG. 3B shows an anterior view of the dorsal hand plate in an apparatus according to embodiments of the disclosure;

FIG. 4 shows a side view of the digit and dorsal hand plate in an apparatus according to embodiments of the disclosure;

FIG. 5 shows a dorsal view of a digit with a mechanical fastener in a first position in an apparatus according to embodiments of the disclosure;

FIG. 6 shows a side view of a digit with a mechanical fastener in a second position according to embodiments of the disclosure;

FIG. 7A shows a front view of a gear set configured for implementation in an apparatus according to embodiments of the disclosure;

FIG. 7B shows a front view of further embodiments of a gear set configured for implementation in an apparatus according to embodiments of the disclosure;

FIG. 7C shows a front view of additional embodiments of a gear set configured for implementation in an apparatus according to embodiments of the disclosure.

It is noted that the drawings of the disclosure are not to scale. The drawings are intended to depict only some aspects of the disclosure, and therefore should not be considered as limiting the scope of the disclosure. In the drawings, like numbering represents like elements between the drawings.

### DETAILED DESCRIPTION

In the following description, reference is made to the accompanying drawings that form a part thereof, and in which is shown by way of illustration specific illustrative embodiments in which the present teachings may be practiced. These embodiments are described in sufficient detail to enable those skilled in the art to practice the present teachings, and it is to be understood that other embodiments may be used and that changes may be made without departing from the scope of the present teachings. The following description is, therefore, merely illustrative.

It will be understood that when an element is referred to as being “on” or “over” another element, it may be directly on the other element or intervening elements may also be present. In contrast, when an element is referred to as being “directly on” or “directly over” another element, there may be no intervening elements present. It will also be understood that when an element is referred to as being “connected” or “coupled” to another element, it may be directly connected or coupled to the other element or intervening elements may be present. In contrast, when an element is referred to as being “directly connected” or “directly coupled” to another element, there are no intervening elements present.

Reference in the specification to “one embodiment” or “an embodiment” of the present disclosure, as well as other variations thereof, means that a particular feature, structure, characteristic, and so forth described in connection with the embodiment is included in at least one embodiment of the present disclosure. Thus, the phrases “in one embodiment” or “in an embodiment,” as well as any other variations appearing in various places throughout the specification are not necessarily all referring to the same embodiment. It is to be appreciated that the use of any of the following “/,” “and/or,” and “at least one of,” for example, in the cases of “A/B,” “A and/or B” and “at least one of A and B,” is intended to encompass the selection of the first listed option (A) only, or the selection of the second listed option (B) only, or the selection of both options (A and B). As a further example, in the cases of “A, B, and/or C” and “at least one of A, B, and C,” such phrasing is intended to encompass the first listed option (A) only, or the selection of the second listed option (B) only, or the selection of the third listed option (C) only, or the selection of the first and the second listed options (A and B), or the selection of the first and third listed options (A and C) only, or the selection of the second and third listed options (B and C) only, or the selection of all three options (A and B and C). This may be extended, as readily apparent by one of ordinary skill in the art, for as many items listed.

Embodiments of the disclosure provide an apparatus for engaging certain projectiles (e.g., a bowling ball, a soccer ball, a basketball, rachet and disc). Embodiments of the disclosure provide stability to a user while holding a projectile, e.g., by allowing the user to control various aspects of the projectile's release (including axis tilt, revolution rate, release position, etc.), and prevent damage to the projectile by avoiding technical errors (e.g. rolling over the finger holes in bowling). The apparatus is adjustable between multiple positions, e.g., to facilitate a natural grip and release motion, thereby restoring a user's control of the projectile (and thus performance) without additional competitive advantages. Further, the apparatus is operable to replicate the movement of any digit of the hand and can be customized for articulation according to the specific needs and preferences of different users.

Embodiments discussed herein can be customized to fit the needs of the user. For instance, embodiments of the disclosure can be adapted for a user who has a partial finger amputation by anatomically shaping the digit to match the contours of a remaining portion of the finger and mechanically coupling it securely to any remaining portion(s) of the user's finger. In other embodiments, the apparatus can be adapted for a user who has a full finger amputation, e.g., by anatomically shaping the digit to match the user's counterpart (i.e., missing) digit and by affixing it securely to the residual limb. In further embodiments the apparatus is adjustable in length to replicate the natural range of motion and articulation of a finger. This increases comfort, stability, and performance during interaction between the apparatus and projectile. Additionally, the form-fitting nature of the prosthetic finger enhances users'comfort and promotes movement similar to that of the digit or limb lost by the user, without enhancing the ability of a user beyond what may be achieved with a non-artificial limb.

Embodiments of the disclosure can be provided in a static form and/or a dynamic form. In a static form, the apparatus operably engages a projectile(s) and an articulating mechanism can adjust the digit's position before then locking into a position, facilitating a natural grip and release motion. In dynamic form, the digit continually adjusts the articulating mechanism based on a force applied to the projectile, enabling controlled release of the projectile.

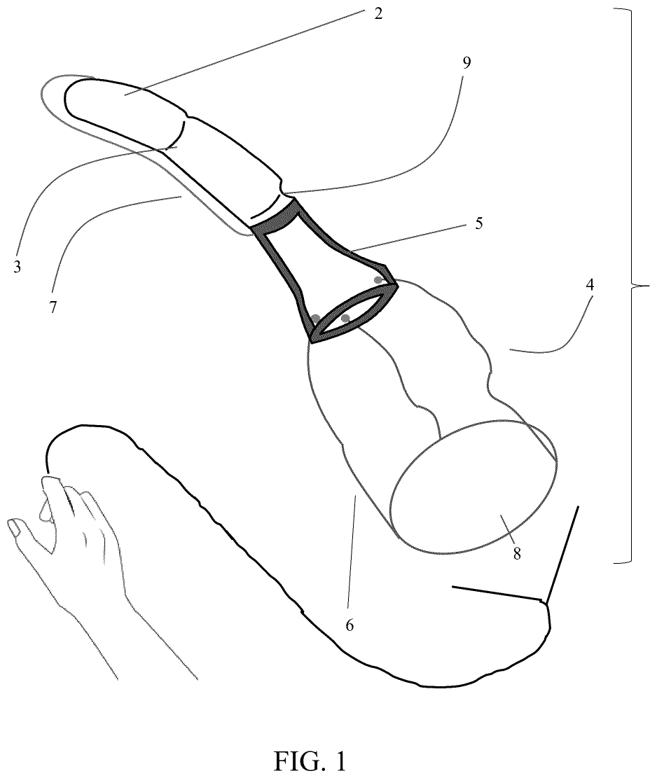

FIG. 1 shows a perspective view of apparatus 10 adapted for use with a partial amputation. A partial amputation refers to a user having at least a portion of a phalanx left on a digit after an amputation. Phalanx or phalanges (plural) refer to the small bones that exist in human toes and fingers. There are three phalanges, the proximal, middle, and distal phalanx, in most fingers and toes. However, the thumb and large toe have only two phalanges. For non-thumb/big toe digits, the proximal and middle phalanx are connected by a distal interphalangeal joint and the middle phalanx is connected to the proximal phalanx by a proximal interphalangeal joint. Thumbs and big toes have a proximal interphalangeal joint that connects the distal phalanx to the proximal phalanx. Therefore, regarding non-thumb or big toe digits, a partial amputation may be the physical or operative loss of a portion of the distal phalanx, the entire distal phalanx, the distal phalanx and a portion of the middle phalanx, the entire distal and middle phalanx or the entire distal and middle phalanx and a portion of the proximal phalanx. Additionally, for thumbs or big toes a partial amputation may be the removal of a portion of the distal phalanx, the entire distal phalanx, or the distal phalanx and a portion of the proximal phalanx.

Apparatus 10 may include a digit 1, which in turn may include a plurality of phalanx 2, set of spacers 3, an adjustable fastener 4, sleeve 5, anchoring wires 6, deformable material 7, mechanical fastener 8, and an articulating mechanism 9. Embodiments of digit 1 may comprise a plurality of phalanx 2 interdigitated with a set of spacers 3 and connected to an articulating mechanism 9 and adjustable fastener 4 by any known or later developed method. Digit 1 engages with the projectile(s) 11 and allows the user to impart a force on the projectile(s) 11, similar to that of a natural finger but not so much as to give the user an unfair advantage. In some embodiments, digit 1 can be adapted to a particular user and/or projectile(s) 11, such as adjusting the length and size of the plurality of phalanx 2 and the set of spacers 3 to be proportional to the users damaged counterpart digit. For example, digit 1 may be adapted for a user who has lost the entire distal and middle phalanx of their right pointer finger. In such an example, digit 1 could have two phalanx 2 that are approximate proportional to the lost distal and middle phalanx, respectively, and plurality of phalanx 2 would be connected by a spacer 3 proportional to the lost distal interphalangeal joint.

The plurality of phalanx 2, of digit 1, may be interdigitated with a set of spacers 3 made of a durable and lightweight material such as silicone or thermoplastic elastomer. In embodiments the set of spacers 3 act as replacements for interphalangeal joints with the number of spacers, size, and shape being determined by the counterpart amputated digit of the user. Additionally, in some embodiments the articulating mechanism 9 may be disposed within the spacers 3. The plurality of phalanx 2 are each made of a durable and lightweight material such as silicone or thermoplastic elastomer. In some embodiments the plurality of phalanx 2 may be adjusted by articulating mechanism 9, to set positions or change positions continually based on a particular user and/or projectile(s) 11 in order to replicate the articulation of a counterpart digit. Further details of articulating mechanism 9 and manners of coupling to phalanx 2 are discussed herein.

In some embodiments, digit 1 can also have a deformable material 7 externally coupled to one or more of the plurality of phalanx 2. The deformable material 7 may be a silicone elastomer, or any other material, known or later developed, that is a textured surface able compress under pressure, to replicate a finger's response. For example, as shown in FIG. 1, the deformable material 7 may cover a side of distal phalanx 2, a middle phalanx 2, and a spacer 3 connecting the plurality of phalanx 2. In this example, the deformable material 7 could be silicone rubber manufactured to have a stiffness specific to the amount of grip required by the user or projectile(s) 11, such as adjusting the stiffness based on the weight of a bowling ball and the speed of the bowler's swing.

In some embodiments, the articulating mechanism 9 is connected to the plurality of phalanx 2 and the set of spacers 3 and in some embodiments the articulating mechanism 9 is disposed within the set of spacers 3. Depending on the embodiment the articulating mechanism 9 may be either static and/or dynamic in nature. The articulating mechanism 9 adjust the position of the plurality of phalanx 2 to fit the user's needs, and in some embodiments are, but are not limited to, a gear set, screw, or piston. An embodiment of the articulating mechanism 9, shown in FIG. 1, is disposed within the set of spacers 3 and is in static form. In the embodiment, articulating mechanism 9 adjusts the angle of the plurality of phalanx 2, relative to the adjustable fastener 8, before use and then locks the plurality of phalanx 2 into position for use.

Apparatus 10 connects digit 1 to the user's residual limb 21 using an adjustable fastener 4, which may be any known or later developed method for coupling. In the embodiment shown in FIG. 1 digit 1 is secured to the remaining portion of the user's residual limb 21 using an adjustable fastener 4 comprising a sleeve 5, mechanical fastener 8 and a set of anchoring wires 6. Sleeve 5 is configured to be inserted over the partial amputation site and connected to a set of anchoring wires 6 at predetermined anchoring points. Sleeve 5 may be any known or later developed elastic and lightweight material. For example, sleeve 5 may be nylon configured to mate with a counterpart digit having only a proximal phalanx remaining. Anchoring wires 6, made of durable elastic material such as, but not limited to, rubber and are attached to the mechanical fastener 8 which secures the apparatus to the user's residual limb 21. The length of the anchoring wires 6 can be adjusted to fit digit 1 on the user. Additionally, the mechanical fastener 8 may be any mechanism which can operably couple to a user's residual limb, whether currently known (e.g. wrist band, adhesive, strap) or later developed. For example, in FIG. 1, sleeve 5 mates with a user's proximal phalanx and sleeve 5 is connected to a wrist band 8 via three anchoring wires 6 attached to the sleeve 5 at anchoring points. The wrist band 8 is secured around the user's residual limb 21 and the anchoring wires 6 are adjusted in length based on the user's comfort.

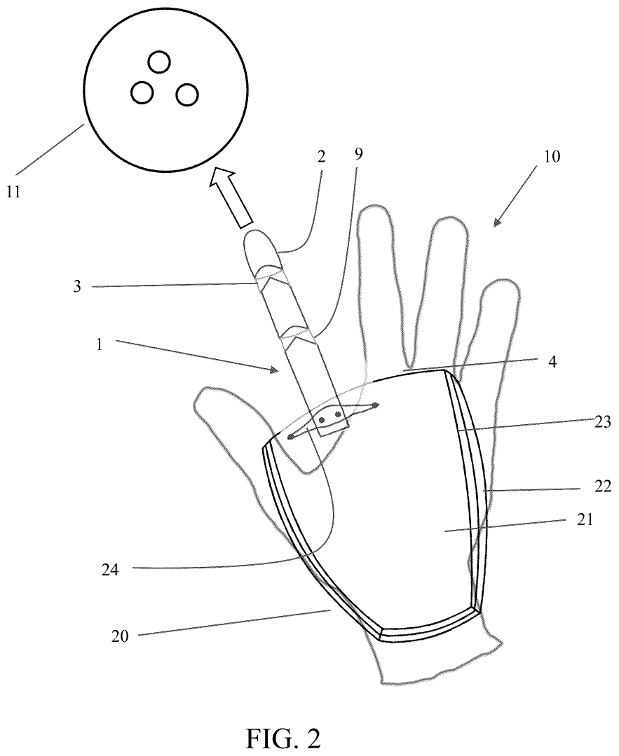

Another embodiment, as shown in FIG. 2 can affix to a partial amputation or full amputation according to embodiments of the disclosure. A full amputation refers to the complete physical or operative loss of a user's finger. Therefore, a full amputation would be the removal of the distal, middle, and proximal phalanx for non-thumb/big toe digits and the distal and proximal phalanx for the thumb and big toe. In such implementations, apparatus 10 may include a digit 1, a plurality of phalanx 2, a set of spacers 3, articulating mechanism 9, an adjustable fastener 4, a dorsal hand plate 20, a first plate 22, a second plate 26, a well 23, and a mechanical fastener 8. As in other embodiments the digit 1 as shown in FIG. 2 includes a plurality of phalanx 2 connected by a set of spacers 3. The number of phalanx 2 and the set of spacers 3 can be adjusted in length, size, and/or number to meet the needs of the user or for specific projectile(s) 11 (e.g., bowling ball, disc, football). In further embodiments the length and size of the phalanx 2 and spacers 3 are proportional to the user's damaged counterpart digit or the user's other remaining digits (e.g. a digit 1 for a fully amputated right pointer finger whose dimensions are based on the left pointer finger). In an example of apparatus 10, digit 1 compensates for the full amputation of the user's right pointer finger. In such an example, digit 1 could have three phalanx 2 that are approximate to the size and shape of the lost distal, middle, and proximal phalanx respectively, and plurality of phalanx 2 would be connected by a spacer 3 having the same approximate size and shape as the lost distal and proximal interphalangeal joints.

In some embodiments, such as shown in FIG. 2, adjustable fastener 4 connects digit 1 to the user's residual limb 21 using a dorsal hand plate 20 including a first plate 22, a second plate 26, a well 23, and a mechanical fastener 8 (not shown). First plate 22 couples to digit 1 and can be coupled by any known or later developed method. In the example of FIG. 2 a brace 24 is used. The first plate 22 sits above and is operatively engaged with a second plate 26 which is housed within a well 23 and may be coupled to the well 23 by any known or later developed method. In some embodiments, first plate 22 is operatively coupled to second plate 26 by a set of interlocked teeth 25, allowing the user to adjust the position of digit 1 for engaging with projectiles 11 and mimic the length of the amputated finger. Further, some embodiments may have second plate 26 coupled to the well 23 using a slide rail 27, allowing the second plate 26 to adjust its resting position within the well 23, changing the length of the apparatus. Additionally, the first plate 22, second plate 26, and well 23 may be made of a durable and lightweight material either known or later developed. For example, the first plate 22, second plate 26, and well 23 may be all comprised of a thermoplastic.

The adjustable fastener 4 is secured to the user's residual limb via a mechanical fastener 8. Mechanical fastener 8 may be any mechanism which can operably couple to a user's residual limb 21, whether existing now (e.g. wrist band, adhesive, strap) or later developed. In one example, a dorsal hand plate 20 via a glove 8 inserted over the user's residual limb and coupled to second plate 26.

As shown in FIG. 2, apparatus 10 enables a user to change the position of the digit 1 before use (static form) and/or can change the position of the digit 1 while in use, in response to the amount of force applied to the projectile 11 (dynamic form). In some embodiments, static form includes articulating mechanism 9 disposed within the set of spacers 3. Articulating mechanism 9 can adjust the position of the plurality of phalanx 2 to fit the user's needs, and in some embodiments are, but are not limited to, a gear set, screw, or piston. For example, articulating mechanism 9 is a gear set 30 able to lock in place, allowing the user to adjust the position of the plurality of phalanx 2 based on comfort and the projectile(s) 11.

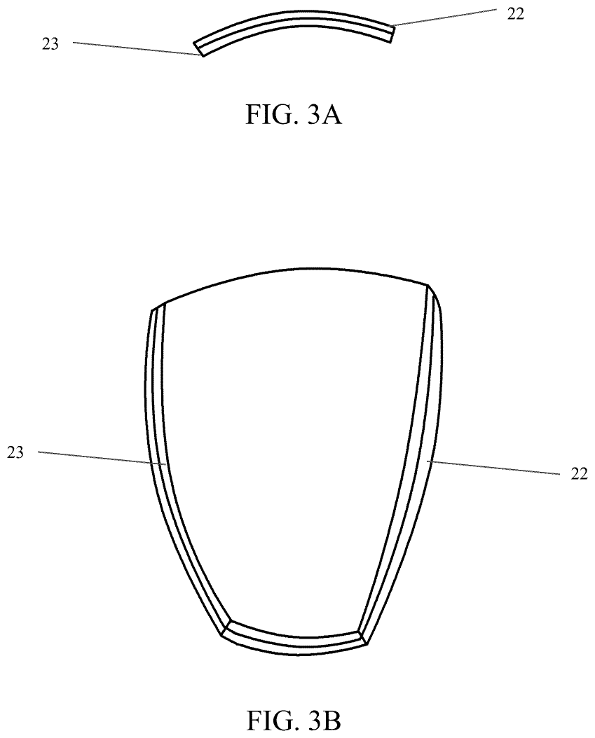

FIGS. 3A-B show a side and dorsal view, respectively, of the dorsal hand plate 20 in an embodiment of apparatus 10. The figures show first plate 22 operatively engaged with second plate 26 and positioned within well 23. The first plate 22, second plate 26, and well 23 may be curved to fit the user's residual limb 21. Additionally, in some embodiments first plate 22, second plate 26 and well 23 are sized and shaped to fit, or otherwise have complementary shapes relative to, the users residual limb 21. For example, a user having an amputated right pointer finger may have dorsal hand plate 20 dimensioned to rest on the residual limb's 21 dorsal side and angled to position digit 1 adjacent to the amputation site.

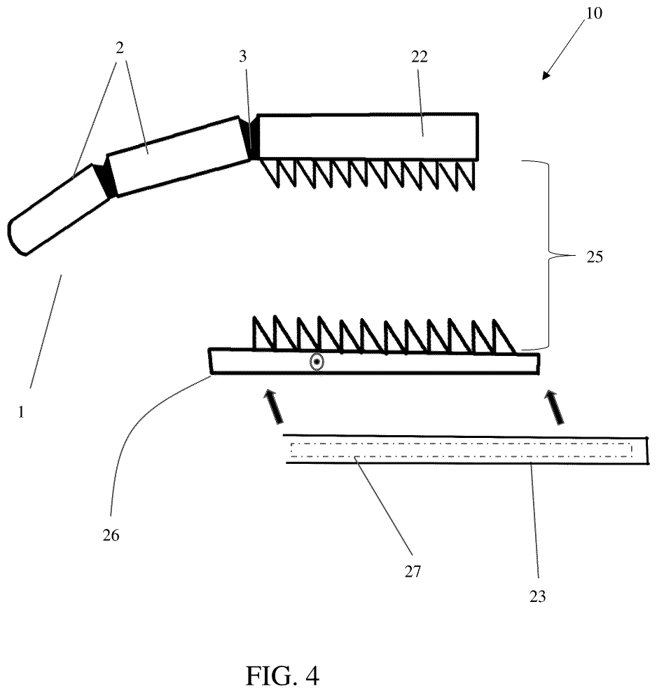

FIG. 4. shows a side view of an embodiment including a digit 1 and a dorsal hand plate 20. Digit 1, similar to other embodiments, includes a plurality of phalanx 2 interdigitated with a set of spacers 3. Digit 1 is coupled to the first plate 22 of the dorsal hand plate 20. The first plate 22 operatively engages with second plate 26, which is housed within well 23. First plate 22 and second plate 26 may be operatively engaged using any known or later developed method. For example, as shown in FIG. 4, the first plate 22 operatively engages with the second plate 26 by a set of interlocking teeth 25 externally coupled on the outside of first plate 22 and second plate 26, with interlocked teeth 25 opposed to each other. Interlocking teeth 25 allow the length of the digit to be adjusted (e.g., increased and/or decreased) to increase the user's comfort and ability to interact with projectile 11. Further, the second plate 26 is housed within well 23 and can be coupled by any known or later developed method. In an embodiment the second plate 26 is coupled to the well 23 using a slide rail 27, allowing the second plate 26 to adjust its resting position within the well 23, changing the length of apparatus 10.

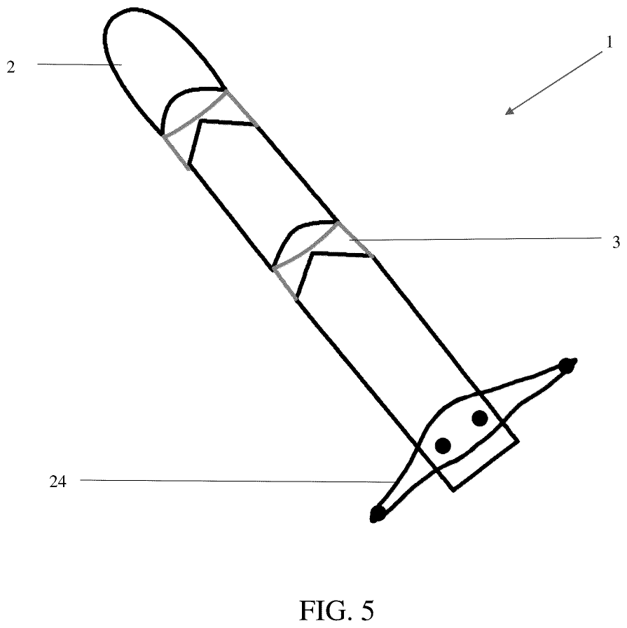

FIG. 5 shows a top-down view of another embodiment of digit 1 in apparatus 10 according to the disclosure. The digit 1 in this embodiment contains three phalanx 2 interdigitated with a set of spacers 3. In some embodiments the shape, size, and/or number of plurality of phalanx 2 will be proportional to the phalanx of the damaged counterpart digit of the user, while shape, size, and/or number of the set of spacers 3 will be proportional to the interphalangeal joints of the damaged counterpart digit of the user. Digit 1 may couple to adjustable fastener 4, by any known or later developed method. For example, as shown in FIG. 5. digit 1 may be three phalanx 2 structurally corresponding to the lost distal, middle, and proximal phalanx respectively, and plurality of phalanx 2 would be connected by a spacer 3 proportional to the lost distal and proximal interphalangeal joints. In this example, digit 1 may be coupled to the adjustable fastener 4 via a brace 24, as shown in FIG. 5.

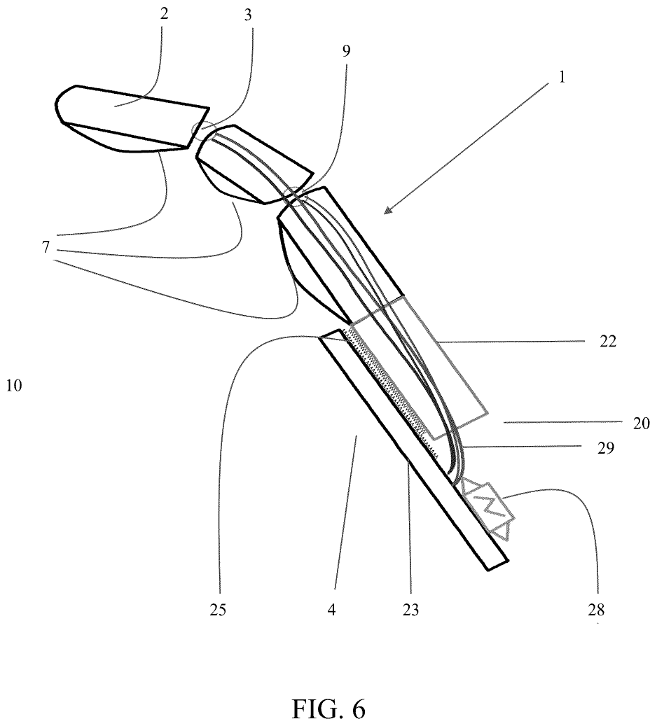

Apparatus 10, as shown in FIG. 6 can be adapted for use with a partial amputation or full amputation and may be configured to move in a dynamic form, according to various embodiments of the disclosure. Apparatus 10 may include a digit 1, a plurality of phalanx 2, set of spacers 3, an adjustable fastener 4, deformable material 7, an articulating mechanism 9, dorsal hand plate 20, first plate 22, second plate 26, well 23, a set of interlocked teeth 25, a controller 28, and wires 29. Digit 1 may have different shapes or sizes based on a user's amputation being partial or full, as previously discussed and shown in FIGS. 1 and 2. Additionally, in some embodiments deformable material 7 may be externally coupled to digit 1. In the example shown in FIG. 6, deformable material is coupled to a side of plurality of phalanx 2 but does not cover the set of spacers 3. Digit 1 may be coupled to adjustable fastener 4, by any known or later developed method. For example, the digit 1 shown in FIG. 6 is coupled to the first plate 22 of the dorsal hand plate 20, via adhesive on a side of one of the plurality of phalanx 2. Further, in FIG. 6, the dorsal hand plate 20 includes a first plate 22, a well 23, a second plate 26 disposed within the well 23, and a set of interlocking teeth 25. As previously described, well 23 allows the second plate 26 to lock into various positions, changing the overall length of the adjustable fastener 4 and apparatus 10. Further, the set of interlocking teeth 25 allows the apparatus 10 to adjust the position of digit 1.

The embodiment shown in FIG. 6 is a dynamic form of apparatus 10. That is, unlike the static form, in which the articulating mechanism 9 can set the position of the plurality of phalanx 2 before using a projectile 11, some embodiments of the articulating mechanism 9 will continually adjust the position of the plurality of phalanx 2. In some embodiment the articulating mechanism 9 is disposed within spacer 3 and adjusts the plurality of phalanx 2 based on the amount of force applied to projectile 11 by digit 1. For example, articulating mechanism 9 may be gear sets 30 within spacers 3, that upon contact with a projectile(s) will adjust the position of the proximal, middle, and distal phalanx 2. In this example, the gear set 30 within the spacer 3, corresponding to the distal interphalangeal joint, will apply a rotational force on the phalanx 2, equivalent to the users damaged distal phalanx, the force being a counter force equal to that applied by the phalanx 2 to the projectile 11. Additionally, the force applied by the articulating mechanisms 9 may be the same or different for each phalanx 2 of the plurality of phalanx 2.

In some embodiments the adjustments made by the articulating mechanism 9 are determined by a controller 28. Controller 28 may be any computing device or mechanical device able to perform a desired function given certain input variables. For example, controller 28 may be able to determine the number of rotations a gear set 30 within spacer 3 needs in response to a input of force applied to a projectile. Embodiments of controller 28 include wherein the controller 28 is coupled to second plate 26, by any known or later developed method. Further, controller 28 optionally may be connected to articulating mechanism 9 via a set of wires 29, or any other known or later developed way such as wireless data couplings. In some embodiments wires 29 connect to controller 28 and then are coupled to the first plate 22 and the plurality of phalanx 2, in order to connect with the articulating mechanism 9 disposed within the spacers 3. For example, wires 29 may extend from controller 28 and pass through a cavity in first plate 22 and through a cavity in the plurality of phalanx 2, in order to protect the wires 29 from damage and to minimize interference with engaging projectile(s) 11. In this example, controller 28 would send signals to articulating mechanism 9, via wires 29, determining the position of the plurality of phalanx 2.

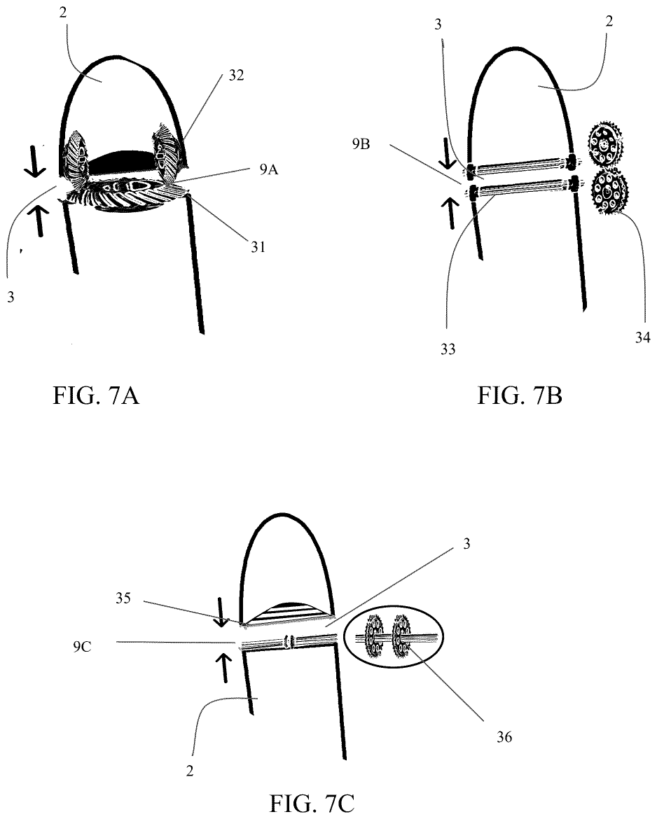

FIGS. 7A-C show multiple embodiments of an articulating mechanism 9. In some embodiments the articulating mechanism 9 can be, but is not limited to, a gear set, screw, or piston. FIGS. 7A-C shows embodiments in which the articulating mechanism 9 is a gear set 30 disposed within spacer 3. The gear sets 30 may be adjusted and locked into a position before using a projectile 11 (static form) or may rotate continually throughout use of projectile 11 based on force applied by the plurality of phalanx 2 on the projectile 11 (dynamic form). FIG. 7A shows an embodiment in which gear set 30 are bevel gears. In an example, a phalanx 2, corresponding to the user's damaged distal phalanx, changes position based on a bevel gear set 30. In this example, the bevel gear set has a horizontal gear 31 and two vertical gears 32. In the example, controller 28 turns horizontal gear 31 based on the amount of force applied to phalanx 2, horizontal gear 31 then turns vertical gear(s) 32 which are coupled to the plurality of phalanx 2. Thus, turning vertical gears 32 will adjust the position of phalanx 2 corresponding to the distal phalanx.

FIG. 7B shows an embodiment in which the gear set 30 is implemented with a set of shafts 33 connected to spur gears 34. In an example, phalanx 2, corresponding to the user's damaged distal phalanx, changes position based on spur gears 34 connected to two shafts 33. In this example, each shaft 33 and corresponding spur gears 34 are connected to a phalanx 2 of the plurality of phalanx and controller 28 turns the shafts 33 based on the amount of force applied to a respective phalanx 2.

FIG. 7C again shows an embodiment in which the gear set 30 is a rack 35 and pinion 36 within spacers 3. In an example, rack 35 is coupled to a side of a phalanx 2, corresponding to the users damaged distal phalanx, while pinion 36 is coupled to a side of a phalanx 2, corresponding to the user's damaged middle phalanx, and operatively engages with rack 35. In this example, controller 28 turns pinion 36 based on the amount of force applied to phalanx 2, thus changing the position of the phalanx 2, corresponding to the user's damaged distal phalanx. In further example, gear set 30 may be implemented via any of various other currently known or later developed mechanical couplings, bearings, transmissions, etc.

The apparatus 10, as described, allows users with damaged digits to regain any lost ability to use projectiles, without also enhancing such ability relative to uninjured competitors. Apparatus 10 provides stability and control to the user while using a projectile, similar to that of a natural digit, without imparting additional competitive advantages. Further, apparatus 10 is adjustable based on the user's comfort and damaged digit as well as to the projectile(s) being used.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about”, “approximately” and “substantially”, is not to be limited to the precise value specified. In at least some instances, the approximating language may correspond to the precision of an instrument for measuring the value. Here and throughout the specification and claims, range limitations may be combined and/or interchanged. Such ranges are identified and include all the sub-ranges contained therein unless context or language indicates otherwise. “Approximately” as applied to a particular endpoint of a range applies to both endpoints and, unless otherwise dependent on the precision of the instrument measuring the value, may indicate +/−10% of the stated endpoint value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described to best explain the principles of the disclosure and the practical application, and to enable others of ordinary skill in the art to understand the disclosure for various embodiments with various modifications as are suited to the particular use contemplated.

What is claimed is:

1. An apparatus comprising:
a digit configured to operably engage a projectile, the digit including:
a plurality of phalanx interdigitated with a respective set of spacers and configured to replicate the articulation of a counterpart digit, and
a deformable material externally coupled to one of the plurality of phalanx configured to replicate a grip of the counterpart digit on the projectile;
an adjustable fastener coupled to the digit and configured to securing the digit to a limb; and
an articulating mechanism operatively coupled to the plurality of phalanx and configured to adjust a positional configuration of the digit.

2. The apparatus of claim 1, wherein a length of the digit is proportional to a partially amputated finger of a patient.

3. The apparatus of claim 2, wherein the adjustable fastener includes:
a mechanical fastener configured to secure the digit to the limb, and
a sleeve configured to mate with the partially amputated finger of the patient and connect to the mechanical fastener through a set of anchoring wires.

4. The apparatus of claim 1, wherein a length of the digit is proportional to a fully amputated finger of a patient.

5. The apparatus of claim 4, where the adjustable fastener is a dorsal hand plate including:
a first plate coupled to the digit and operatively engaging a second plate,
a well having the second plate therein; and
a mechanical fastener coupling the well to the limb.

6. The apparatus of claim 5, wherein a set of interlocked teeth couple the first plate to the second plate.

7. The apparatus of claim 1, wherein the deformable material is coupled to an exterior of at least two phalanx of the plurality of phalanx.

8. An apparatus comprising:
a digit configured to operably engage a projectile, the digit including:
a plurality of phalanx interdigitated with a respective set of spacers and configured to replicate the articulation of a counterpart digit, and
a deformable material externally coupled to one of the plurality of phalanx configured to replicate a grip of the counterpart digit on the projectile;
an adjustable fastener coupled to the digit and configured to securing the digit to a limb;
an articulating mechanism operatively coupled to the plurality of phalanx and configured to adjust a positional configuration of the digit; and
a controller connected to the articulating mechanism and configured to operate the articulating mechanism based on a force applied to the projectile.

9. The apparatus of claim 8, wherein a length of the digit is proportional to a partially amputated finger of a patient.

10. The apparatus of claim 9, wherein the adjustable fastener includes:
a mechanical fastener configured to secure the digit to the limb, and
a sleeve configured to mate with the partially amputated finger of the patient and connect to the mechanical fastener through a set of anchoring wires.

11. The apparatus of claim 8, The apparatus of claim 1, wherein a length of the digit is proportional to a fully amputated finger of a patient.

12. The apparatus of claim 11, wherein the adjustable fastener is a dorsal hand plate including:
a first plate coupled to the digit and operatively engaging a second plate,
a well having the second plate therein; and
a mechanical fastener coupling the well to the limb.

13. The apparatus of claim 12, wherein a set of interlocked teeth couple the first plate to the second plate.

14. The apparatus of claim 8, wherein the deformable material is coupled to an exterior of at least two phalanx of the plurality of phalanx.

15. The apparatus of claim 8, wherein the articulating mechanism comprises a gear set disposed within the set of spacers.

16. A method of manufacturing an apparatus, the method comprising:
forming a digit configured to operably engage a projectile, the digit including:
a plurality of phalanx interdigitated with a respective set of spacers and configured to replicate the articulation of a counterpart digit, and
a deformable material externally coupled to one of the plurality of phalanx configured to replicate a grip of the counterpart digit on the projectile;
forming an adjustable fastener coupled to the digit and configured to securing the digit to a limb; and
forming an articulating mechanism operatively coupled to the plurality of phalanx and configured to adjust a positional configuration of the digit.

17. The method of claim 16, wherein a length of the digit is formed proportionally to a partially amputated finger of a patient.

18. The method of claim 17, wherein the adjustable fastener includes:
a mechanical fastener configured to secure the digit to the limb, and
a sleeve configured to mate with the partially amputated finger of the patient and connect to the mechanical fastener through a set of anchoring wires.

19. The method of claim 16, wherein a length of the digit is formed proportionally to a fully amputated finger of a patient.

20. The method of claim 19, wherein the adjustable fastener is a dorsal hand plate including:
a first plate coupled to the digit and operatively engaging a second plate, a well having the second plate therein; and
a mechanical fastener coupling the well to the limb.
